(19) United States
(12) Patent Application Publication
(10) Pub. No.: US 2013/0231393 A1
(43) Pub. Date: Sep. 5, 2013

(54) USE OF SQUARAMIDE IN THE PREVENTION AND/OR TREATMENT OF ROSACEA

(71) Applicant: Jérôme Aubert, Grasse (FR)

(72) Inventor: Jérôme Aubert, Grasse (FR)

(73) Assignee: GALDERMA RESEARCH & DEVELOPMENT, Biot (FR)

(21) Appl. No.: 13/807,675

(22) PCT Filed: Jun. 29, 2011
(86) PCT No.: PCT/EP11/60953

§ 371 Date: Apr. 3, 2013

(30) Foreign Application Priority Data
Jun. 29, 2010 (FR) 1055245

(51) Int. Cl. C07C 237/44

(52) U.S. Cl. CPC C07C 237/44
USPC 514/619; 564/167

(57) ABSTRACT

A compound of formula (I):

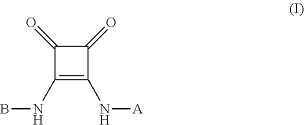

or a pharmaceutically acceptable salt, a pharmaceutically acceptable solvate, or hydrate thereof are described. Also described, is the use of such a compound, salt, solvate or hydrate in the preparation of a medicament intended to treat rosacea.

### TECHNICAL FIELD OF THE INVENTION

The present invention relates to a novel use of a compound of the formula (I) or of one of its pharmaceutically acceptable salts, pharmaceutically acceptable solvates, or hydrates, in the preparation of a medicament intended to prevent and/or treat rosacea.

### BACKGROUND OF THE INVENTION

Rosacea is a common, chronic and progressive inflammatory dermatosis related to vascular relaxation. It mainly affects the central part of the face and is characterized by redness of the face or hot flushes, facial erythema, papules, pustules, telangiectasia and sometimes an eye injury, also called ocular rosacea. In serious cases, particularly in men, the soft tissue of the nose may swell and produce a bulbous swelling known as rhinophyma.

Rosacea generally occurs from the ages of 25 and 70, and it is much more common in individuals with a light complexion. It affects more particularly women, although this condition is generally more serious in men. Rosacea is chronic and persists for years with periods of exacerbation and remission.

Rosacea was originally called ‘acne rosacea’ because its papules (points of slight raising of the skin) and its inflammatory pustules (pus scabs) greatly resemble those of common acne.

The result of this facial vascular anomaly is a permanent oedema of the dermis which may accompany an increased colonization with Demodex folliculorum, a parasite usually found in the follicles of the face.

Many factors may also be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

According to the National Rosacea Society, rosacea may be classified into four subtypes plus one variant (Erythematotelangiectatic rosacea, papulopustular, phymatous, eyepiece and a variant called granulomatous rosacea).

These subtypes are listed bellow.

Subtype 1: erythematotelangiectatic rosacea.

Subtype 1 is characterized by flushing and persistent central facial erythema. Telangiectases are common but not essential for the diagnosis. Central facial edema, burning sensations and scales are also reported symptoms. Conventionnaly, the patients have erythrosis spasms due to the sudden dilation of the arterioles of the face, which then take on a congestive, red appearance. These spasms can be triggered by the emotions, meals and temperature changes.

Subtype 2: papulopustular rosacea.

Subtype 2 is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Burning sensations may be reported by patients with papulopustular rosacea. This subtype has often been seen after or in combination with subtype 1, including the presence of telangiectases. The telangiectases may be obscured by persistent erythema, papules, or pustules. Some patients may also have cheeks and forehead edema.

Subtype 3: Phymatous rosacea.

Subtype 3 includes thickening skin, irregular surface nodularities, and enlargement. Rhinophyma is the most common presentation, but phymatous rosacea may occur in other locations, including the chin, forehead, cheeks, and ears. Patients with this subtype may also have enlarged and prominent follicle openings. This subtype has frequently been observed after or in combination with subtypes 1 or 2, including persistent erythema, telangiectases, papules, and pustules. In the case of rhinophyma, these additional stigmata may be especially pronounced in the nasal area.

Subtype 4: ocular rosacea.

The diagnosis of ocular rosacea should be considered when a patient's eyes have one or more of the following signs and symptoms : watery or bloodshot appearance (interpalpebral conjunctival hyperemia), foreign body sensation, burning, dryness, itching, light sensitivity, blurred vision, telangiectasia of the conjunctiva and lidmargin, or lid and periocular erythema, blepharitis, conjunctivitis, Meibomian gland dysfunction. These signs or symptoms occur before, during or after the onset of cutaneous signs. Ocular rosacea is most frequently diagnosed when cutaneous signs and symptoms of rosacea are also present. However, skin signs and symptoms are not prerequisite to the diagnosis, and limited studies suggest that ocular signs and symptoms may occur before cutaneous manifestations in up to 20% of patients with ocular rosacea.

Granulomatous rosacea.

There is also a granulomatous variant of rosacea, characterized by yellow , brown or red, indurated papules or nodules, and monomorphic damage in papules. Other signs of rosacea may also occur.

Of course, the pathological manifestations of rosacea vary depending on the subtype of the disease. However patients may have characteristics of different subtypes at the same time. It is also known that the disease does not necessarily progress from a subtype to another (Wilkin et al., 2002, J. AM. Acad. Dermatol. Vol. 46, pages 584-587).

Conventionally, rosacea is treated orally or topically with antibiotics such as tetracyclines, erythromycin or clindamycin, but also with salicylic acid, antifungal agents, steroids, metronidazole or with isotretinoin in severe cases, or even with anti-infectious agents such as azelaic acid.

As disclosed in application PCT/EP2010/069896, rosacea is characterized by induction of expression of the following cytokines and chemokines: interleukin 8 (IL-8), CXCL1, CXCL2, CXCL 3 and CXCL5, CXCR1 receptor, receptor CXCR2.

IL-8 (CXCL-8) is a member of the CXC chemokine family that plays a primordial role in the trafficking of neutrophils to the site of inflammation (For a review see Busch-Petersen J.;Curr Top Med Chem. 2006;6(13):1345-52). IL-8 has also been described to play a roles (i) in endothelial cells activation (Transactivation of Vascular Endothelial Growth Factor Receptor-2 by Interleukin-8 (IL-8/CXCL8) Is Required for IL-8/CXCL8-induced Endothelial Permeability. D Melissa L. et al (2007) Molecular Biology of the Cell Vol. 18, 5014-5023); (ii) in vascular permability increase (Transactivation of Vascular Endothelial Growth Factor Receptor-2 by Interleukin-8 (IL-8/CXCL8) Is Required for IL-8/CXCL8-induced Endothelial Permeability. D Melissa L. et al (2007) Molecular Biology of the Cell Vol. 18, 5014-5023,) and (iii) in neovascularization (IL-8 Directly Enhanced Endothelial Cell Survival, Proliferation, and Matrix Metalloproteinases Production and Regulated Angiogenesis. Aihua Li et al, The Journal of Immunology, 2003, 170: 3369-3376; Autocrine role of IL8 in induction of EC proliferation survival, migration and MMP2 production and angiogenesis Aihua Li et al , Angiogenesis, 2005, 8:63-71; The CXC Chemokine Receptor 2, CXCR2, Is the Putative Receptor for ELR1 CXC Chemokine-Induced Angiogenic Activity. Christina L. Addison et al The Journal of Immunology, 2000, 165: 5269-5277.).

Two chemokine G-Protein-coupled, seven-transmembrane receptors (CXCR1 and CXCR2) are known to be specifically activated by IL-8. While CXCR2 binds with high affinity to IL-8 and other chemokines such as CXCL6, CXCL5, CXCL2, CXCL3 and CXCL1, CXCR1 binds only IL-8.

Therefore, potent dual antagonists of CXCR1 and CXCR2 may hold promise in limiting the deleterious effects of the inflammatory response and angiogenesis mediated by IL-8 and associated chemokines, and thereby prevent scarring.

Patent application WO02/083624 disclosed a family of squaramides as IL-8 antagonistes and used for treatment of diseases mediated by that chemokine such as atopic dermataitis, osteoarthritis, pulmonary diseases or disorders, acne.

Patent Application WO 08/079122 disclosed a family of squaramides as IL-8 antagonistes and used for treatment of an inflammatory or allergic condition or obstructive airway desease.

It is now found, unexpectedly, that some of the compounds of this patent application are CXCR1 and CXCR2 antagonists and capable of being active in preventing and/or treating skin disorders and preferably rosacea.

### SUMMARY OF THE INVENTION

The present invention relates to a novel use of compounds in the preparation of a medicament intented fo prevent and/or treat rosacea.

More specifically, the present invention relate to the compound of formula (I):

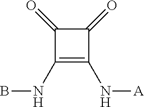

wherein A is selected from:

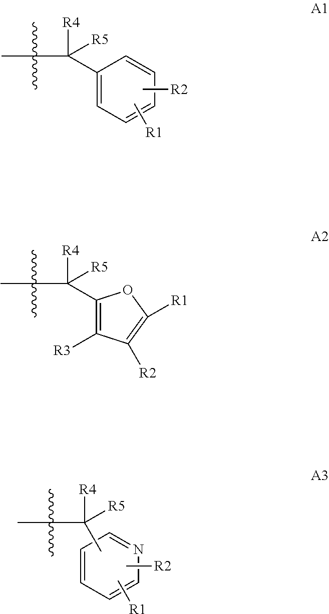

and B is selected from:

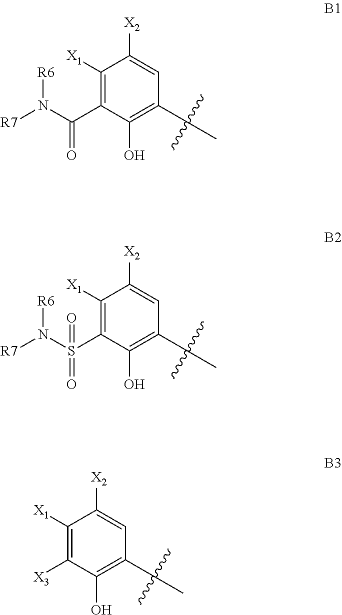

where

R1, R2, R3 are independently selected from hydrognene, halogen or C1-C5 alkyl groups;

R4, R5 are selected from hydrogene or C1-C5 alkyl groups; R4 and R5 can be joined together to form a C3-C6 cycloalkyl group; R5 can also be CF3;

R6 and R7 are independently selected from hydrognene or C1-C5 alkyl groups; or can be joined together to form a 6 membered heterocycloalkyl ring, that could be substituted with one heteroatom e.g. oxygen to form a morpholine ring, preferably to form a C3-C6 heterocycloalkyl group ; and

X1, X2, X3 are selected from : hydrogen, cyanide, fluoride, chloride, bromide, trifluoromethyl, or nitro;

or its pharmaceutically acceptable salts, its pharmaceutically acceptable solvates or their hydrates in the preparation of a medicament intended to prevent and/or treat rosacea.

In a preferred embodiment of the present invention, B has the formula:

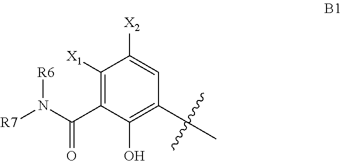

where R6 and R7 are independently selected from C1-C5 alkyl groups, and X1 and X2 are hydrogen.

In another embodiment, A is selected from:

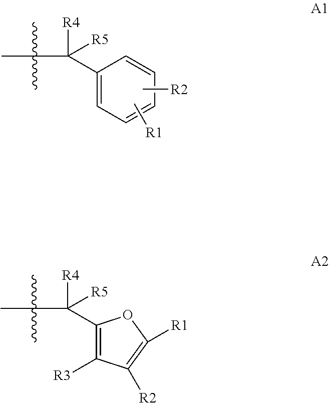

where R4 is an hydrogen, R5 is a C1-C5 alkyl group, and R1, R2 and R3 are independently selected from the group consisting of hydrogen, halogen or C1-C5 group.

In a preferred embodiment, the present invention relates to the use of a compound of formula (I), wherein B has the formula:

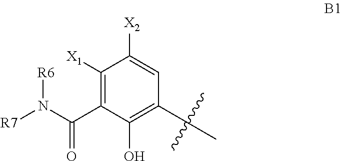

where R6 and R7 are methyl groups, X1 and X2 are hydrogen,

and where A has the formula:

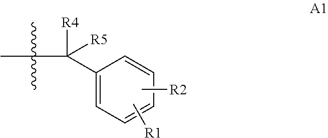

where R1 is a methyl group, R2, R3 and R4 are hydrogen, and where R5 is a C1-C5 alkyl group;

or its pharmaceutically acceptable salts, its pharmaceutically acceptable solvates or their hydrates in the preparation of a medicament intended to prevent and/or treat rosacea.

In a preferred embodiment, the compound of formula (I) is selected from a group of molecules disclosed in the detailed description section, preferentially 2-Hydroxy-N,N-dimethyl-3-{2-[(R)-1-(5-methyl-furan-2-yl)-propylamino]-3,4-dioxo-cyclobut-1-enylamino}-benzamide; or its pharmaceutically acceptable salts, its pharmaceutically acceptable solvates or their hydrates.

In another embodiment, the present invention relates to the use of a compound of formula (I) for the preparation of a medicament appropriate for topical administration.

In another aspect of the present invention, there are disclosed methods for preventing and/or treating rosacea, comprising administering to a patient in need thereof, the effective amount of at least one of any compound described herein; or its pharmaceutically acceptable salts, its pharmaceutically acceptable solvates or their hydrates.

In a further aspect of the present invention, there are provided processes for the preparation of any compound described herein

### DETAILED DESCRIPTION OF THE INVENTION

The present invention thus relates to the use of compound of following formula (I):

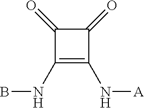

wherein

A is selected from:

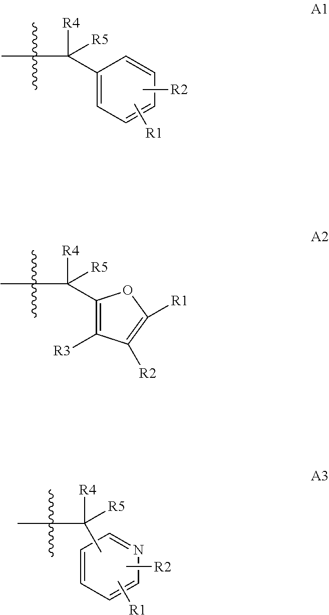

B is selected from:

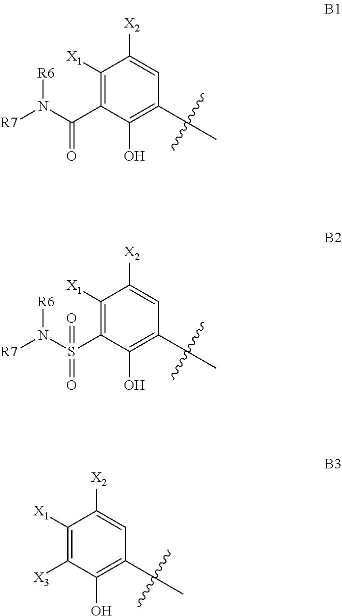

R1, R2, R3 are selected from hydrogene, halogen or C1-C5 alkyl groups;

R4, R5 are selected from hydrogene or C1-C5 alkyl groups; R4 and R5 can be joined together to form a C3-C6 cycloalkyl group; R5 can also be CF3;

R6 and R7 are selected from C1-C5 alkyl group; or R6 and R7 can be joined together to form a 6 membered heterocycloalkyl ring, that could be substituted with one heteroatom e.g. oxygen to form a morpholine ring, preferably to form a C3-C6 heterocycloalkyl group;

X1, X2, X3 are selected from : hydrogen, cyanide, fluoride, chloride, bromide, trifluoromethyl, or nitro;

or its pharmaceutically acceptable salts, its pharmaceutically acceptable solvates or their hydrates, in the preparation of a medicament intended to prevent and/or treat rosacea.

In a preferred embodiment, the present invention relates to the use of compound of formula (I) wherein B is selected from:

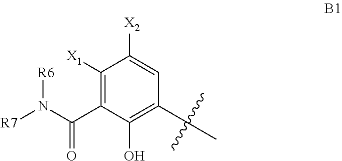

where R6 and R7 are selected from a C1-C5 alkyl group,

X1 and X2 are hydrogen;

and A is selected from:

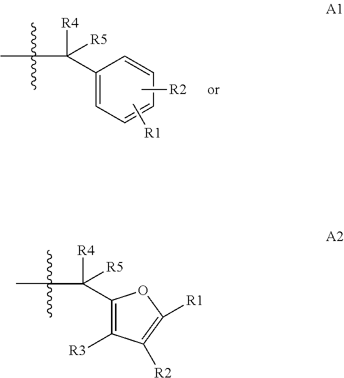

wherein R4 is an hydrogen, R5 is a C-C5 alkyl group, R1, R2 and R3 are selected from hydrogen or a C1-C5 alkyl group.

In a preferred embodiment, the present invention relates to the use of compound of formula (I) wherein B is selected from:

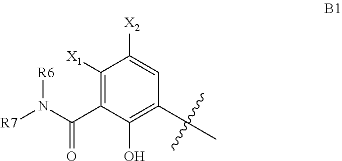

where R6 and R7 are methyl groups,

X1 and X2 are hydrogen;

and A is selected from:

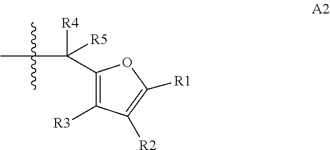

wherein R4 is an hydrogen, R5 is a C1-C5 alkyl group,

R1 is a methyl group,

R2 and R3 are hydrogen.

In the context of the invention alkyl group means a saturated hydrocarbon chain with from 1 to 10 carbon atoms and preferably from 1 to 4 carbon atoms.

According to the present invention, a cycloalkyl is a chain saturated hydrocarbon, cyclic, comprising from 3 to 7 carbon atoms, preferably comprising from 3 to 6 carbon atoms.

Accordingly, an heterocycloalkyl is a chain saturated hydrocarbon, cyclic, comprising comprising from 3 to 7 carbon atoms, preferably comprising from 3 to 6 carbon atoms and comprising at least one heteroatom, preferably 1 to 4 heteroatoms selected from nitrogen, oxygen, and sulfur.

In a particular embodiment the heterocycloalkyl is a C3-C6 heterocycloalkyl .

The invention relates more specifically to the use of 2-Hydroxy-N,N-dimethyl-3-{2-[(R)-1-(5-methyl-furan-2-yl)-propylamino]-3,4-dioxo-cyclobut-1-enylamino}-benzamide also kown as SCH 527123 or one of their pharmaceutically acceptable salts, pharmaceutically acceptable solvates or hydrates in the preparation of a medicament intended to prevent and/or treat rosacea

In another aspect of the present invention provides a compound of formula (I) selected from the group consisting of:

1: 2-Hydroxy-N,N-dimethyl-3-{2-[(R)-1-(5-methyl-furan-2-yl)-propylamino]-3,4-dioxo-cyclobut-1-enylamino}-benzamide

2: 2-Hydroxy-N,N-dimethyl-3-{2-[(4-methyl-furan-2-ylmethyl)-amino]-3,4-dioxo-cyclobut-1-enylamino}-benzamide

3: 2-Hydroxy-N,N-dimethyl-3-{2-[(4-isopropyl-furan-2-ylmethyl)-amino]-3,4-dioxo-cyclobut-1-enylamino}-benzamide

4: 3-{2-[(R)-1-(4-Ethyl-furan-2-yl)ethylamino]-3,4-dioxo-cyclobut-1-enylamino}-2-hydroxy-N,N-dimethyl-benzamide

5: 3-{2-[(R)-1-(4-Ethyl-furan-2-yl)ethylamino]-3,4-dioxo-cyclobut-1-enylamino}-2-hydroxy-N,N-dimethyl-benzamide

6: 3-[2-((R)-1-Furan-2-yl-ethylamino)-3,4-dioxo-cyclobut-1-enylamino]-2-hydroxy-N,N-dimethyl-benzamide

7: 3-[3,4-Dioxo-2-((R)-1-phenyl-ethylamino)-cyclobut-1-enylamino]-2-hydroxy-N,N-dimethyl-benzamide

8: 3-[3,4-Dioxo-2-((R)-1-phenyl-ethylamino)-cyclobut-1-enylamino]-2-hydroxy-N,N-dimethyl-benzamide

9: 3-[3,4-Dioxo-2-((R)-1-phenyl-propylamino)-cyclobut-1-enylamino]-2-hydroxy-N,N-dimethyl-benzamide

10: 3-[3,4-Dioxo-2-((R)-1-pyridin-2-yl-propylamino)-cyclobut-1-enylamino]-2-hydroxy-N,N-dimethyl-benzamide

11: 3-[3,4-Dioxo-2-((R)-1-pyridin-3-yl-propylamino)-cyclobut-1-enylamino]-2-hydroxy-N,N-dimethyl-benzamide

12: 3-[3,4-Dioxo-2-((R)-1-pyridin-4-yl-propylamino)-cyclobut-1-enylamino]-2-hydroxy-N,N-dimethyl-benzamide

13: 3-(2-Hydroxy-phenylamino)-4-((R)-1-phenyl-propylamino)-cyclobut-3-ene-1,2-dione

14: 3-(5-Fluoro-2-hydroxy-phenylamino)-4-((R)-1-phenyl-propylamino)-cyclobut-3-ene-1,2-dione

15: 3-(4-Fluoro-2-hydroxy-phenylamino)-4-((R)-1-phenyl-propylamino)-cyclobut-3-ene-1,2-dione

16: 3-[3,4-Dioxo-2-((R)-1-phenyl-propylamino)-cyclobut-1-enylamino]-N,N-diethyl-2-hydroxy-benzamide

17: 3-[3,4-Dioxo-2-((R)-1-phenyl-propylamino)-cyclobut-1-enylamino]-N,N-diethyl-2-hydroxy-benzenesulfonamide

18: 2-Hydroxy-N,N-dimethyl-3-{2-[(R)-1-(5-methyl-furan-2-yl)-propylamino]-3,4-dioxo-cyclobut-1-enylamino}-benzenesulfonamide

19: 3-[3,4-Dioxo-2-(1-phenyl-cyclopropylamino)-cyclobut-1-enylamino]-2-hydroxy-N,N-dimethyl-benzamide

20: 3-[2-(1-Furan-2-yl-1-methyl-ethylamino)-3,4-dioxo-cyclobut-1-enylamino]-2-hydroxy-N,N-dimethyl-benzamide

21: 3-{2-[(Furan-2-ylmethyl)-amino]-3,4-dioxo-cyclobut-1-enylamino}-2-hydroxy-N,N-dimethyl-benzamide

22: 3-{2-[(R)-1-(5-Chloro-furan-2-yl)-propylamino]-3,4-dioxo-cyclobut-1-enylamino}-2-hydroxy-N,N-dimethyl-benzamide

23: 3-{3,4-Dioxo-2-[(S)-2,2,2-trifluoro-1-(5-methyl-furan-2-yl)-ethylamino]-cyclobut-1-enylamino}-2-hydroxy-N,N-dimethyl-benzamide

A compound of the present invention may exist in the form of isomers and mixtures thereof; e.g. optical isomers, diastereoisomers. A compound of the present invention may contain asymmetric carbon atoms and may exist in the form of enantiomers or diastereoisomers and mixtures thereof, e.g. racemates.

The salts of the compounds of formula (I) according to the invention comprise salts with organic or inorganic bases, for example alkali metal salts, such as lithium, sodium or potassium salts.

The term “hydrate of a compound of formula (I)” is understood to mean a combination of this compound with one or more water molecules.

The term “solvate of a compound of formula (I)” is understood to mean the combination resulting from the attachment of a solvent to the crystals of compound of formula (I) which are formed in the presence of this solvent.

The compounds of formula (I) is in particular synthesized as described in Application WO 02/083624.

The commercially available or synthesized by skilled man in the art arylamines (B-NH2) are condensed with the commercially available diethyl squarate to give the corresponding aminoethoxysquarate product (step 1). Subsequent condensation of this intermediate with the amino compounds A-NH2, commercially available or prepared by skilled man in the art (step 2) gives the final product of general formula I.

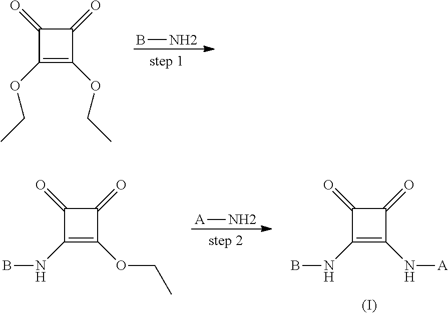

### EXAMPLES

### Example 1

### Comparative Analysis in a Neurogenic Skin Inflammation Model

This example provides a comparative analysis of compounds of invention in a neurogenic skin inflammation model.

Neurogenic inflammation is a well-defined process by which inflammation is triggered by the nervous system. Different stimulations are recognized as a trigger of neurogenic inflammation like hot beverage, environmental temperature changed, spicy food, sun-exposure. In the face, these stimuli are felt by sensory fibers originating from the trigeminal sensory nerves, which contain several neuropeptides such as substance P (SP), neurokinin A (NA), and calcitonin gene-related peptide (CGRP). These neuropeptides are well known to have potent vasodilatory and vascular leakage properties. Histologically, neurogenic inflammation is characterized by an oedema, vasodilation, and infiltrates of leukocytes. In the skin, neurogenic inflammation results in redness, swelling, heat, and localized pain. These characteristics are found in Rosacea and especially in Subtype I or erythematotelangiectatic rosacea. The release of vasoactive neuropeptides is thought to result in a sustained neurogenic inflammation within the skin which causes a transient (flush) or permanent facial erythema and oedema. Moreover, neuropeptides such as SP may act on mast cells to induce histamine release. Histamine may then exacerbates the inflammatory response by increasing the permeability of the capillaries to white blood cells and increasing vasodilation.

The capsaicin, the pungent ingredient from peppers, is widely used as a tool in sensory neuron biology to induce neurogenic inflammation. Indeed, capsaicin activates the vanilloid type 1 receptor (TRPV1), which is a non-selective cation channel that is structurally related to members of the TRP family of ion channels. Binding of capsaicin to the TRPV1 receptor provokes neurogenic inflammation through depolarization of a subpopulation of primary sensory neurons leading to the release of neuropeptides (CGRP, SP . . . ). This receptor is also activated by increases in temperature, suggesting that it functions as a transducer of painful thermal stimuli in vivo.

The animal model of neurogenic skin inflammation was induced by a unique topical application of resiniferatoxin (RTX), 0.03% on the back of hairless SKH1 mice. RTX is an ultrapotent capsaicin analog and RTX exposure evokes neuropeptides release. Most of these neuropeptides have vasodilatory properties and one component of RTX-induced neurogenic inflammation is the dermal vasodilation. In the present RTX-model, the objective assessment of the skin vasodilation was evaluated by laser Doppler perfusion imaging. The increase in skin blood flow provoked by RTX application to the skin is to a large extent antagonized by a TRPV1 receptor antagonist, the capsazepine, (CZP) at 5% (relative area of vasodilation, AUC decreased by 13% and * * * p<0.001 for the kinetic). SCH527123, a CXCR1/2 antagonist, was evaluated in this model at 2%.

Material and Method:
Measurement of RTX-Induced Vasodilation

Adult female hairless SKH1 mice aged 7-9 weeks are obtained from Charles River (France). Anaesthesia with ketamine/xylazine is performed 10 minutes before treatment and body temperature is maintained at 35° C.-37° C. using an automated heating pad. A skin area of 1.8×1.5 cm on the back of the mouse is divided in two regions of interest. One region is treated with the vehicle, the other one with RTX 0.03%. The compounds to be tested are applied 4 minutes before the single topical application of resiniferatoxin (RTX) on one region; the other region was used for vehicule application. Each mouse is its own control. The skin blood perfusion is evaluated every 2 minutes on the back of the mouse with a Laser Doppler perfusion imaging PIM3 (Perimed, France). Prior to treatment, scans are performed and measurements of blood perfusion change are made over 20 minutes. At the end of the study, mice are euthanized by cervical dislocation. Images are analysed and the yield of two parameters, the erythematous surface and the blood perfusion intensity ratio, is calculated as a relative area of vasodilation (mm²). For each mouse, the value of the control area is substracted from the value of the treated area. Results are expressed as mean±SEM. The statistical analysis performed is based on a repeated analysis of variance (P) of the relative area of vasodilation. This analysis allowed the comparison of the group effect on the kinetic of the vasodilation.

Results:

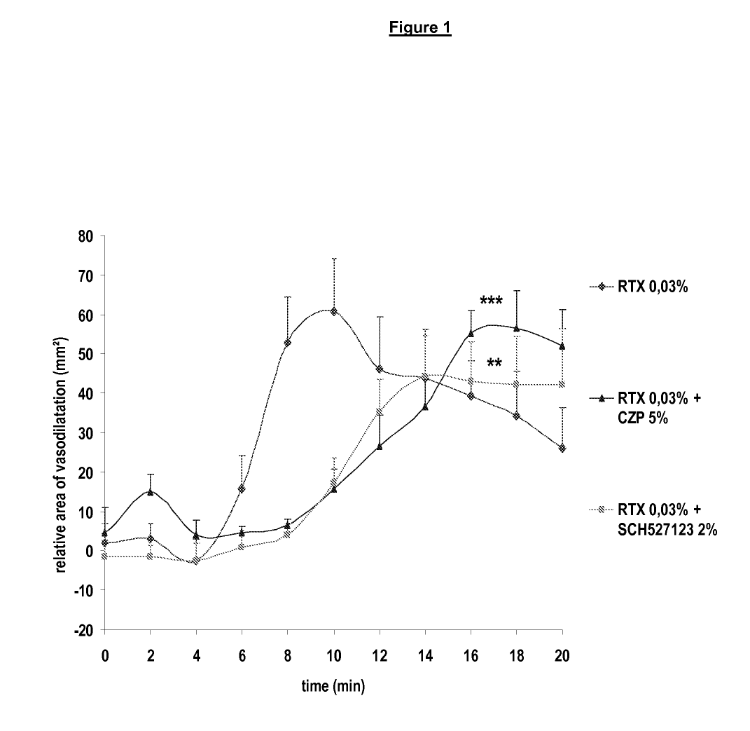

The results are shown in FIG. 1: SCH527123 used at the dose of 2% inhibited the vasodilation induced by the topical application of RTX 0.03% on the back of SKH1 mice (relative area of vasodilation, AUC decreased by 31% and **p<0.01 for the kinetic).

This example demonstrate that a compound of invention is effective against vasodilatation in a neurogenic skin inflammation model.

### Example 2

### Activity Data

This exemple provides comparative activity data for inhibition of CXCR1 and CXCR2. The data are obtained with followinfg assay:

### β-Arrestin Recruitment Assay.

Antagonism of CXCL8-Stimulated β-Arrestin2 Recruitment. Activation of CXCR2 (PathHunter HEK293 hCXCR2 b-arrestin) or CXCR1 (U2OS hCXCR1 b-arrestin) by CXCL8 has been shown to lead to recruitment of β-arrestin (Richardson et al., 2003). To monitor direct interaction of CXCR2 or CXCR1 with β-arrestin2, we used a β-arrestin2 recruitment assay for CXCR2 or CXCR1 based on enzyme complementation of β-galactosidase (Olson and Eglen, 2007), as established by DiscoveRx Corporation. Stimulation of both cell lines with CXCL8 induces β-arrestin2 recruitment, as indicated by a significant fold increase. All CXCR2-antagonists are tested in dose-depedant manner (firsty dose 10 μM).

PathHunter HEK293-hCXCR2 b-arrestin cells or U2OS hCXCR1 b-arrestin cells (both from DiscoveRx Corporation) were plated out overnight at 10,000 cells/well (384-well format) in 50 μl of Opti MEM I+1% bovine serum albumin. Cells were treated with CXR2 antogonist or vehicule (Opti MEM I) and CXCL-8 at the same time for 90 min.; thereafter, 25 μl of PathHunter Detection Reagents (DiscoveRx Corporation) was added. After an incubation of 60 min at room temperature, β-galactosidase-induced luminescence upon β-arrestin-CXCR1/2 interaction was measured in an TopCount Reader (Perkin Elmer). Functional data were evaluated by a nonlinear curve fitting procedure using XLFit4 (IDBS).

| | IC50 | IC50 |
|---|---|---|
| Compound name | CXCR2 | CXCR1 |
| 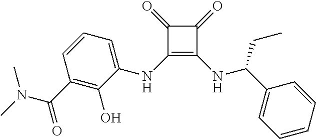 [SMILES: CC[C@@H](Nc1c(Nc2cccc(C(=O)N(C)C)c2O)c(=O)c1=O)c1ccccc1] 3-[3,4-Dioxo-2-((R)-1-phenyl-propylamino)-cyclobut-1- enylamino]-2-hydroxy-N,N-dimethyl-benzamide | 7 nM | 148 nM |
| 2-Hydroxy-N,N-dimethyl-3-{2-[(R)-1-(5-methyl- | 9 nM | 49 nM |
| furan-2-yl)-propylamino]-3,4-dioxo-cyclobut-1- | | |
| enylamino}-benzamide | | |
| (SCH527123) | | |

This example confirms the inhibiting activity on both CXCR1 and CXCR2. However, SCH527123 appears to be more potent on the 2 receptors.

1. A compound of formula (I): 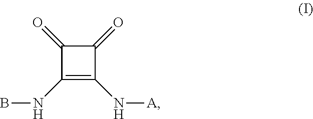
wherein A is: 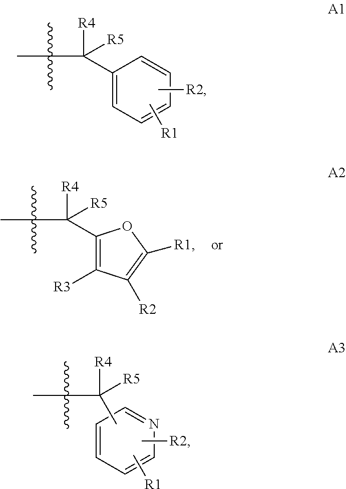
B is: 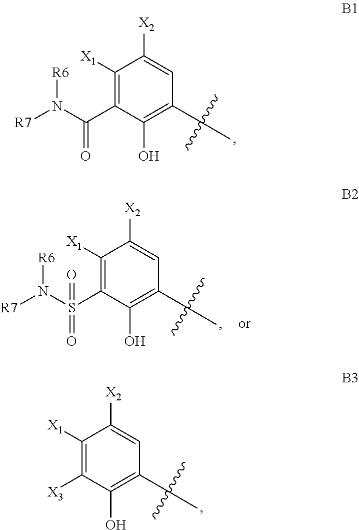
R1, R2, R3 are independently hydrogen, a halogen or a C1-C5 alkyl group;
R4, R5 are hydrogen or a C1-C5 alkyl groups; R4 and R5 can be joined together to form a C3-C6 cycloalkyl group; or R5 can also be CF3;
R6 and R7 are independently hydrogen or a C1-C5 alkyl group; or can be joined together to form a 6 membered heterocycloalkyl ring, that is optionally substituted with one heteroatom; and
X1, X2, X3 are: hydrogen, cyanide, fluoride, chloride, bromide, trifluoromethyl, or nitro;
or a pharmaceutically acceptable salt, a pharmaceutically acceptable solvate or hydrate thereof.

2. The compound of claim 1, wherein R6 and R7 are joined together to form a 6 membered heterocycloalkyl ring, that is substituted with one heteroatom.

3. The compound of claim 2, wherein the heteroatom is an oxygen.

4. The compound of claim 1, wherein B has the formula: 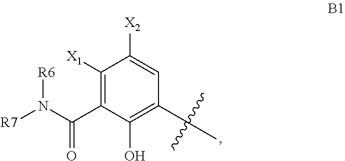
where R6 and R7 are independently a C1-C5 alkyl group, and where X1 and X2 are hydrogen.

5. The compound of claim 1, wherein A is: 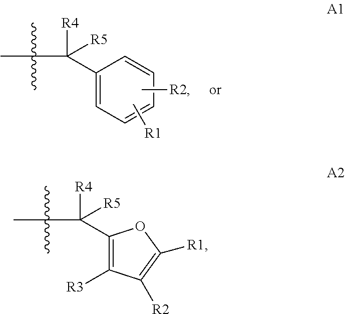
where R4 is hydrogen, R5 is a C1-C5 alkyl group, and where R1, R2 and R3 are independently hydrogen or a C1-C5 group.

6. The compound of claim 1, wherein B has the formula: 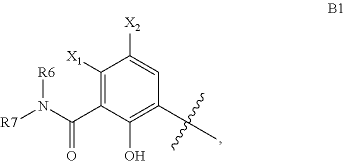
where R6 and R7 are methyl groups, X1 and X2 are hydrogen, and where A has the formula: 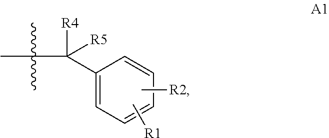
where R1 is a methyl group, R2, R3 and R4 are hydrogen, and where R5 is a C1-C5 alkyl group.

7. The compound of claim 1, wherein the compound of formula (I) is selected from the group consisting of:
1:2-Hydroxy-N,N-dimethyl-3-{2-[(R)-1-(5-methyl-furan-2-yl)-propylamino]-3,4-dioxo-cyclobut-1-enylamino}-benzamide;
2:2-Hydroxy-N,N-dimethyl-3-{2-[(4-methyl-furan-2-ylmethyl)-amino]-3,4-dioxo-cyclobut-1-enylamino}benzamide;
3:2-Hydroxy-N,N-dimethyl-3-{2-[(4-isopropyl-furan-2-ylmethyl)-amino]-3,4-dioxo-cyclobut-1-enylamino}-benzamide;
4:3-{2-[(R)-1-(4-Ethyl-furan-2-yl)-ethylamino]-3,4-dioxo-cyclobut-1-enylamino}-2-hydroxy-N,N-dimethyl-benzamide;
5:3-{2-[(R)-1-(4-Ethyl-furan-2-yl)-ethylamino]-3,4-dioxo-cyclobut-1-enylamino}-2-hydroxy-N,N-dimethyl-benzamide;
6:3-[2-((R)-1-Furan-2-yl-ethylamino)-3,4-dioxo-cyclobut-1-enylamino]-2-hydroxy-N,N-dimethyl-benzamide;
7:3-[3,4-Dioxo-2-((R)-1-phenyl-ethylamino)-cyclobut-1-enylamino]-2-hydroxy-N,N-dimethyl-benzamide;
8:3-[3,4-Dioxo-2-((R)-1-phenyl-ethylamino)-cyclobut-1-enylamino]-2-hydroxy-N,N-dimethyl-benzamide;
9:3-[3,4-Dioxo-2-((R)-1-phenyl-propylamino)-cyclobut-1-enylamino]-2-hydroxy-N,N-dimethyl-benzamide;
10:3-[3,4-Dioxo-2-((R)-1-pyridin-2-yl-propylamino)-cyclobut-1-enylamino]-2-hydroxy-N,N-dimethyl-benzamide;
11:3-[3,4-Dioxo-2-((R)-1-pyridin-3-yl-propylamino)-cyclobut-1-enylamino]-2-hydroxy-N,N-dimethyl-benzamide;
12:3-[3,4-Dioxo-2-((R)-1-pyridin-4-yl-propylamino)-cyclobut-1-enylamino]-2-hydroxy-N,N-dimethyl-benzamide;
13:3-(2-Hydroxy-phenylamino)-4-((R)-1-phenyl-propylamino)-cyclobut-3-ene-1,2- dione;
14:3-(5-Fluoro-2-hydroxy-phenylamino)-4-((R)-1-phenyl-propylamino)-cyclobut-3-ene-1,2-dione;
15:3-(4-Fluoro-2-hydroxy-phenylamino)-4-((R)-1-phenyl-propylamino)-cyclobut-3-ene-1,2-dione;
16:3-[3,4-Dioxo-2-((R)-1-phenyl-propylamino)-cyclobut-1-enylamino]-N,N-diethyl-2-hydroxy-benzamide;
17:3-[3,4-Dioxo-2-((R)-1-phenyl-propylamino)-cyclobut-1-enylamino]-N,N-diethyl-2-hydroxy-benzenesulfonamide;
18:2-Hydroxy-N,N-dimethyl-3-{2-[(R)-1-(5-methyl-furan-2-yl)-propylamino]-3,4-dioxo-cyclobut-1-enylaminol-benzenesulfonamide;
19:3-[3,4-Dioxo-2-(1-phenyl-cyclopropylamino)-cyclobut-1-enylamino]-2-hydroxy-N,N-dimethyl-benzamide;
20:3-[2-(1-Furan-2-yl-1-methyl-ethylamino)-3,4-dioxo-cyclobut-1-enylamino]-2-hydroxy-N,N-dimethyl-benzamide;
21:3-{2-[(Furan-2-ylmethyl)-amino]-3,4-dioxo-cyclobut-1-enylamino}-2-hydroxy-N,N-dimethyl-benzamide;
22:3-{2-[(R)-1-(5-Chloro-furan-2-yl)-propylamino]-3,4-dioxo-cyclobut-1-enylamino}-2-hydroxy-N,N-dimethyl-benzamide;
23:3-{3,4-Dioxo-2-[(S)-2,2,2-trifluoro-1-(5-methyl-furan-2-yl)-ethylamino]-cyclobut-1-enylamino}-2-hydroxy-N,N-dimethyl-benzamide; and
a pharmaceutically acceptable salt, a pharmaceutically acceptable solvate or hydrate thereof.

8. The compound of claim 1, wherein the compound of formula (I) is 2-Hydroxy-N,N-dimethyl-3-{2-[(R)-1-(5-methyl-furan-2-yl)-propylamino]-3,4-dioxo-cyclobut-1-enylamino}-benzamide; or a pharmaceutically acceptable salt, a pharmaceutically acceptable solvate or hydrate thereof.

9. The compound of claim 1, wherein the compound is in the form of an isomer or a mixture thereof.

10. The compound of claim 1, wherein the compound comprises an asymmetric carbon atom and is optionally in the form of an enantiomer, a diastereoisomer or a mixture thereof.

11. The compound of claim 1, wherein the salt is comprised of an organic or inorganic base.

12. The compound of claim 1, wherein the compound is incorporated into a medicament for topical administration.

13. A method of treating rosacea, the method comprising administering to a patient in need thereof an effective amount of at least one compound having the following formula (I): 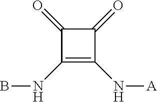
wherein A is: 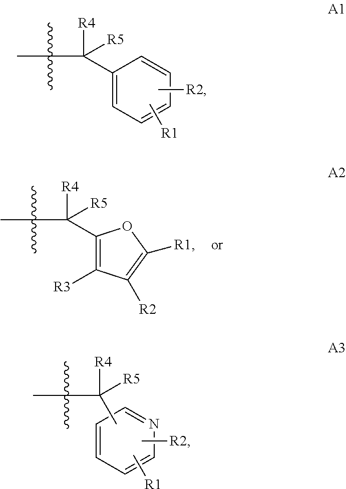
B is: 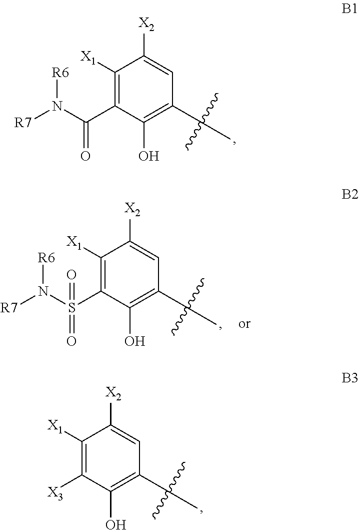
R1, R2, R3 are independently hydrogen, halogen or a C1-C5 alkyl group;
R4, R5 are hydrogen or a C1-C5 alkyl group R4 and R5 can be joined together to form a C3-C6 cycloalkyl group; or R5 can also be CF3;
R6 and R7 are independently hydrogen or a C1-C5 alkyl group; or can be joined together to form a 6 membered heterocycloalkyl ring, that is optionally substituted with one heteroatom; and
X1, X2, X3 are selected from: hydrogen, cyanide, fluoride, chloride, bromide, trifluoromethyl, or nitro;
or its pharmaceutically acceptable salts, its pharmaceutically acceptable solvates or their hydrates.

14. The method of claim 13, wherein effective amount has the formula: 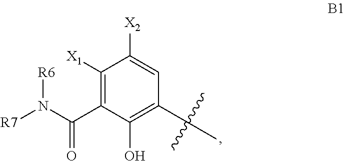
where R6 and R7 are independently a C1-C5 alkyl group, and where X1 and X2 are hydrogen.

15. The method of claim 13, wherein A is: 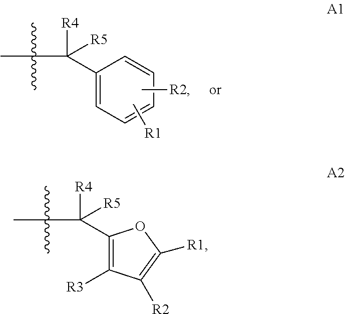
where R4 is a hydrogen, R5 is a C1-C5 alkyl group, and where R1, R2 and R3 are independently hydrogen or a C1-C5 group.

16. The method of claim 13, wherein B has the formula: 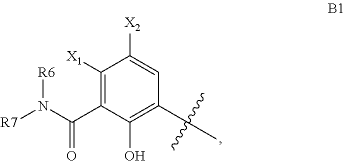
where R6 and R7 are methyl groups, X1 and X2 are hydrogen, and where A has the formula: 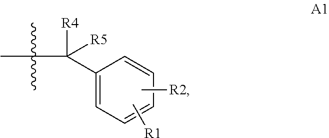
where R1 is a methyl group, R2, R3 and R4 are hydrogen, and where R5 is a C1-C5 alkyl group.

17. The method of claim 13, wherein the compound of formula (I) is selected from the group consisting of:
1:2-Hydroxy-N,N-dimethyl-3-{2-[(R)-1-(5-methyl-furan-2-yl)-propylamino]-3,4-dioxo-cyclobut-1-enylamino}-benzamide;
2:2-Hydroxy-N,N-dimethyl-3-{2-[(4-methyl-furan-2-ylmethyl)-amino]-3,4-dioxo-cyclobut-1-enylaminol-benzamide;
3:2-Hydroxy-N,N-dimethyl-3-{2-[(4-isopropyl-furan-2-ylmethyl)-amino]-3,4-dioxo-cyclobut-1-enylamino}-benzamide;
4:3-{2-[(R)-1-(4-Ethyl-furan-2-yl)-ethylamino]-3,4-dioxo-cyclobut-1-enylamino}-2-hydroxy-N,N-dimethyl-benzamide;
5:3-{2-[(R)-1-(4-Ethyl-furan-2-yl)-ethylamino]-3,4-dioxo-cyclobut-1-enylamino}-2-hydroxy-N,N-dimethyl-benzamide;
6:3-[2-((R)-1-Furan-2-yl-ethylamino)-3,4-dioxo-cyclobut-1-enylamino]-2-hydroxy-N,N-dimethyl-benzamide;
7:3-[3,4-Dioxo-2-((R)-1-phenyl-ethylamino)-cyclobut-1-enylamino]-2-hydroxy-N,N-dimethyl-benzamide;
8:3-[3,4-Dioxo-2-((R)-1-phenyl-ethylamino)-cyclobut-1-enylamino]-2-hydroxy-N,N-dimethyl-benzamide;
9:3-[3,4-Dioxo-2-((R)-1-phenyl-propylamino)-cyclobut-1-enylamino]-2-hydroxy-N,N-dimethyl-benzamide;
10:3-[3,4-Dioxo-2-((R)-1-pyridin-2-yl-propylamino)-cyclobut-1-enylamino]-2-hydroxy-N,N-dimethyl-benzamide;
11:3-[3,4-Dioxo-2-((R)-1-pyridin-3-yl-propylamino)-cyclobut-1-enylamino]-2-hydroxy-N,N-dimethyl-benzamide;
12:3-[3,4-Dioxo-2-((R)-1-pyridin-4-yl-propylamino)-cyclobut-1-enylamino]-2-hydroxy-N,N-dimethyl-benzamide;
13:3-(2-Hydroxy-phenylamino)-4-((R)-1-phenyl-propylamino)-cyclobut-3-ene-1,2-dione
14:3-(5-Fluoro-2-hydroxy-phenylamino)-4-((R)-1-phenyl-propylamino)-cyclobut-3-ene-1,2-dione;
15:3-(4-Fluoro-2-hydroxy-phenylamino)-4-((R)-1-phenyl-propylamino)-cyclobut-3-ene-1,2-dione;
16:3-[3,4-Dioxo-2-((R)-1-phenyl-propylamino)-cyclobut-1-enylamino]-N,N-diethyl-2-hydroxy-benzamide;
17:3-[3,4-Dioxo-2-((R)-1-phenyl-propylamino)-cyclobut-1-enylamino]-N,N-diethyl-2-hydroxy-benzenesulfonamide;
18:2-Hydroxy-N,N-dimethyl-3-{2-[(R)-1-(5-methyl-furan-2-yl)-propylamino]-3,4-dioxo-cyclobut-1-enylamino}-benzenesulfonamide;
19:3-[3,4-Dioxo-2-(1-phenyl-cyclopropylamino)-cyclobut-1-enylamino]-2-hydroxy-N,N-dimethyl-benzamide;
20:3-[2-(1-Furan-2-yl-1-methyl-ethylamino)-3,4-dioxo-cyclobut-1-enylamino]-2-hydroxy-N,N-dimethyl-benzamide;
21:3-{2-[(Furan-2-ylmethyl)-amino]-3,4-dioxo-cyclobut-1-enylamino}-2-hydroxy-N,N-dimethyl-benzamide;
22:3-{2-[(R)-1-(5-Chloro-furan-2-yl)-propylamino]-3,4-dioxo-cyclobut-1-enylamino}-2-hydroxy-N,N-dimethyl-benzamide;
23:3-{3,4-Dioxo-2-[(S)-2,2,2-trifluoro-1-(5-methyl-furan-2-yl)-ethylamino]-cyclobut-1-enylamino}-2-hydroxy-N,N-dimethyl-benzamide; and
a pharmaceutically acceptable salt, a pharmaceutically acceptable solvate or hydrate thereof.

18. The method of claim 17, wherein the compound of formula (I) is 2-Hydroxy-N,N-dimethyl-3-{2-[(R)-1-(5-methyl-furan-2-yl)-propylamino]-3,4-dioxo-cyclobut-1-enylamino}-benzamide; or a pharmaceutically acceptable salt, a pharmaceutically acceptable solvate or hydrate thereof.

19. The compound of claim 1, wherein when R6 and R7 are joined to form a 6 membered heterocycloalkyl ring that is substituted with a heteroatom, the heteroatom is oxygen and a morpholine ring is formed.

20. The compound of claim 1, wherein the R6 and R7 join together to form a C3-C6 heterocycloalkyl group.

21. The compound of claim 9, wherein when the compound is in the form of a mixture, the mixture comprises optical isomers and/or diastereoisomers.

22. The compound of claim 10, wherein when the compound is in the form of a mixture, the mixture is comprised of a racemate.

23. The compound of claim 11, wherein the base is an alkali metal salt.

24. The compound of claim 23, wherein the alkali metal salt is selected from the group consisting of a lithium salt, a sodium salt and a potassium salt.

25. The method of claim 13, wherein when R6 and R7 join together to form a 6 membered heterocycloalkyl ring, the heteroatom used is an oxygen and a morpholine ring is formed.

26. The method of claim 13, wherein the R6 and R7 join together to form a C3-C6 heterocycloalkyl group.
